(19) United States
(12) Patent Application Publication
(10) Pub. No.: US 2019/0159465 A1
(43) Pub. Date: May 30, 2019

(54) BACTERIAL ENDOPHYTE FROM MAIZE AND ASSOCIATED METHODS

(71) Applicant: University of Guelph, Guelph (CA)

(72) Inventors: Manish N. Raizada, Milton (CA); Hanan R. Shehata, Guelph (CA); David Johnston Monje, Ottawa (CA)

(21) Appl. No.: 16/202,862

(22) Filed: Nov. 28, 2018

Related U.S. Application Data
(60) Provisional application No. 62/591,302, filed on Nov. 28, 2017.

(51) Int. Cl. A01N 63/04; C12N 1/20; C12R 1/07

(52) U.S. Cl. CPC A01N 63/04; C12R 1/07; C12N 1/20

(57) ABSTRACT

Described are isolated strains of bacterial endophytes isolated from maize useful for the treatment of plants and associated methods and formulations. The bacterial endophyte may be applied to plants to promote growth. In one embodiment, the bacterial endophyte is an Enterobacter sp. known as 3D9 and deposited as IDAC strain 190917-01.

### RELATED APPLICATIONS

This application claims the benefit of priority of U.S. Provisional Patent Application No. 62/591,302 filed Nov. 28, 2017, the entire contents of which are hereby incorporated by reference.

### FIELD

The present disclosure relates to bacterial endophytes and more specifically to bacterial endophytes isolated from maize.

### BACKGROUND

Cereal crops including maize (Zea mays), wheat (Triticum aestivum L.) and rice (Oryza sativa) are the most cultivated crops worldwide. Plants including cereals host large numbers of microbes (endophytes) that can promote plant health, nutrition and pathogen resistance. Screening microbial collections, with sufficient replicates, for beneficial activities with large, slow-growing cereals is very challenging, as growing these crops requires considerable space, time, labour and cost. A rapid high-throughput in planta system is needed to screen and identify cereal endophytes for beneficial activities so that primary screens can be conducted in planta rather than in vitro only. The use of in planta screening methods allows for the identification of endophytes that are more likely to be useful for commercial agriculture.

There remains a need for novel endophytes useful for promoting plant growth.

### SUMMARY

The present disclosure provides strains of bacterial endophytes isolated from maize and associated methods for the treatment of plants. As demonstrated in the examples, bacterial strain 3D9 is particularly useful for promoting the growth of plants. Bacterial strain 3D9 was observed to increase root biomass as well as shoot biomass, in the absence of external nitrogen fertilizer, in a turfgrass assay relative to an assortment of other bacterial endophytes. 16S ribosomal RNA (rRNA) sequence analysis identified bacterial strain 3D9 as Enterobacter sp. and the strain has been deposited under accession number 190917-01 at the International Depository Authority of Canada located at the National Microbiology Laboratory, Public Health Agency of Canada, 1015 Arlington Street, Winnipeg, Manitoba, R3E 3R2, Canada on Sep. 19, 2017.

Accordingly in one aspect, there is provided an isolated culture of bacterial strain 3D9 deposited as strain IDAC 190917-01. In one embodiment, the bacteria is an Enterobacter sp. In one embodiment, the bacteria colonizes plants as an endophyte. In one embodiment, bacteria 3D9 is isolated from maize, optionally from Zea nicaraguensis.

Also provided is a formulation comprising a bacterial endophyte as described herein, such as bacterial strain 3D9. In one embodiment, the formulation comprises bacterial strain 3D9 deposited as strain IDAC 190917-01 (3D9) and at least one of a carrier, tackifier, microbial stabilizer, fungicide, herbicide, nematicide, insecticide, plant growth regulator, rodenticide, dessicant, nutrient and culture media. In one embodiment, the carrier comprises a surfactant or seed coating agent. Optionally, the formulation may be in liquid form or in solid form. In one embodiment, the formulation is in liquid form and comprises a concentration of 3D9 bacteria of at least 10⁵, 10⁶, 10⁷ or 10⁸ CFU/ml.

Also provided is a synthetic combination comprising a 3D9 bacterial strain as described herein in association with a plant. In one embodiment, the 3D9 bacterial strain is heterologous to the plant. In one embodiment, the bacteria are in association with a seed or seedling. In one embodiment, the bacteria is in association with a seed and the concentration of bacteria is at least 10³ colony-forming units (CFU) per seed, optionally at least 10⁴ or 10⁵ CFU per seed, or between 10⁴ and 10⁷ CFU per seed.

In one embodiment, the plant is an agricultural plant, such as a fruit or vegetable. In one embodiment, the plant is corn. In one embodiment, the plant is corn seed.

In another aspect, there is provided a method of treating a plant using a bacterial endophyte as described herein. For example, in one embodiment, there is provided a method for promoting the growth of a plant comprising contacting the plant with 3D9 bacteria or a formulation comprising 3D9 bacteria. Also provided is a method for promoting nitrogen fixation/bioavailability in a plant, comprising contacting the plant with 3D9 bacteria or a formulation comprising 3D9 bacteria. Different techniques known in the art may be used for contacting the plant with 3D9 bacteria to inoculate the plant with the endophyte. For example, in one embodiment the method comprises applying a spray, a mist, a dip, or a nutrient solution comprising 3D9 bacteria to the plant. In one embodiment, contacting the plant comprises foliar introduction or in-furrow introduction. Also provided is the use of a 3D9 bacteria or formulation thereof as described herein for promoting the growth of a plant and/or promoting nitrogen fixation/bioavailability. In one embodiment, contacting the plant with the bacteria increases biomass of the plant relative to a control plant. In one embodiment, the bacteria increases root biomass of the plant relative to a control plant. In one embodiment, the bacteria improves bioavailable nitrogen in the plant relative to a control plant.

In another aspect, there is provided a method for the production of a bacterial endophyte culture. In one embodiment, method comprises inoculating a substrate with the isolated culture of 3D9 bacteria and incubating the substrate under conditions suitable for bacterial growth. In one embodiment, the substrate is a liquid media culture.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF DRAWINGS

FIG. 1 shows selecting a turfgrass genotype as a model system. (A-E) Germination and growth rate of turfgrass seeds on wet paper towels (water only) after 10 days: (A) annual ryegrass, (B) perennial ryegrass, (C) tall fescue, (D) Bermuda grass and (E) Kentucky bluegrass. (F-J) Growth rate of turfgrass seeds in tubes after 3 weeks: (F) annual ryegrass, (G) perennial ryegrass, (H) tall fescue, (I) Bermuda grass and (J) Kentucky bluegrass. (K-O) Testing for efficiency of seed surface sterilization on R2A agar after 10 days: Sterilization Method 1 is on the left side of each plate and Method 2 is on the right side of each plate: (K) annual ryegrass, (L) perennial ryegrass, (M) tall fescue, (N) Bermuda grass and (O) Kentucky bluegrass. (P-W) Testing growth on different media of: (P-S) annual ryegrass and (T-W) tall fescue; media used were: (P and T) R-2A agar, (Q and U) Bacto-agar, (R and V) sand, and (S and W) Phytagel. (X-Z) Response of annual ryegrass to different nutrient levels after 1 month with respect to (X) shoot biomass and (Y) root biomass, with (Z) corresponding representative pictures (from L-R): plants grown at 0 strength MS, 0.1 strength MS and 0.5 strength MS. In panels X and Y, the error bars represent the standard error of the mean (SEM), while different letters (a, b, c) above each histogram denotes that the means are significantly different from one another.

FIG. 2 shows screening of maize endophytes for growth promotion of annual ryegrass on media without nitrogen. (A) The seed coating methodology for bacterial inoculation. (B) Shoot biomass and (C) root biomass of annual ryegrass at 4 weeks after bacterial inoculation. (D-E) Replicated trials of the growth promotion effect of strain 3D9 on annual ryegrass with respect to: (D) shoot biomass and (E) root biomass. Shown are three independent replicates at 4 weeks after bacterial inoculation. First replicate (n=3 tubes, each with 7 plants) and second and third trials (n=7 tubes, each with 7 plants). The error bars represent the standard error of the mean (SEM). Two asterisks denote that the endophyte treatment is significantly different from the buffer only control at p=0.05, while one asterisk notes that the treatments are significantly different at p=0.10 (student t-test).

FIG. 3 shows dot blot hybridization using a nifH probe to test for nitrogen fixation capacity of strain 3D9. (A) Strain 3D9, (B) positive control (strain USDA110), and (C) negative control (E. coli DH5a).

FIG. 4 shows GlnLux agar plates with corn stems after 6 hours of incubation. Shown are corn stems after 3 weeks of growth, from control uninoculated plants on the left side of each Petri dish, and from 3D9 inoculated plants on the right side of each Petri dish. Shown are three replicates. Images were taken using a CCD camera using a 10 min exposure. Greater light emission denotes higher GlnLux output, indicating higher glutamine content.

FIG. 5 shows a boost in Gln in corn mesocotyl using GlnLux agar after inocultation with 3D9.

FIG. 6 shows a boost in Gln in corn leaves using GlnLux agar after inocultation with 3D9.

FIG. 7 shows the results for control vs plants treated with 3D9 for corn mesocotyl tissue weight and glutamine using the GlnLux assay.

### DETAILED DESCRIPTION

The present disclosure relates to bacterial endophytes isolated from maize. In one embodiment, the bacteria are endophytes capable of promoting activities related to plant growth and/or nutrient acquisition.

As shown in the Examples, turfgrasses were used as a high-throughput model for screening cereal microbes for nutrient-acquisition activities. Turfgrasses consist of 30 species in 20 genera classified as cool-season grasses (including ryegrasses, bluegrasses, fescues) and warm-season grasses (including bermudagrass). Turfgrasses are genetic relatives of cereals, all belonging to the family Poaceae or Gramineae, and hence their endophyte communities may be compatible. However, turfgrasses are much smaller, have faster growth rate, and can be grown in sterilized test tubes on defined media, and under controlled conditions to prevent microbial cross-contamination.

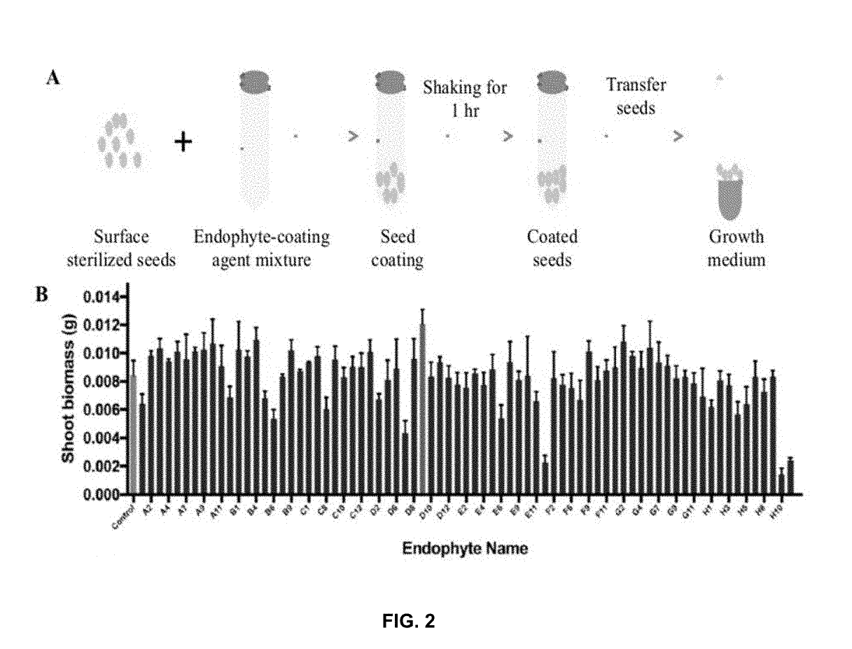

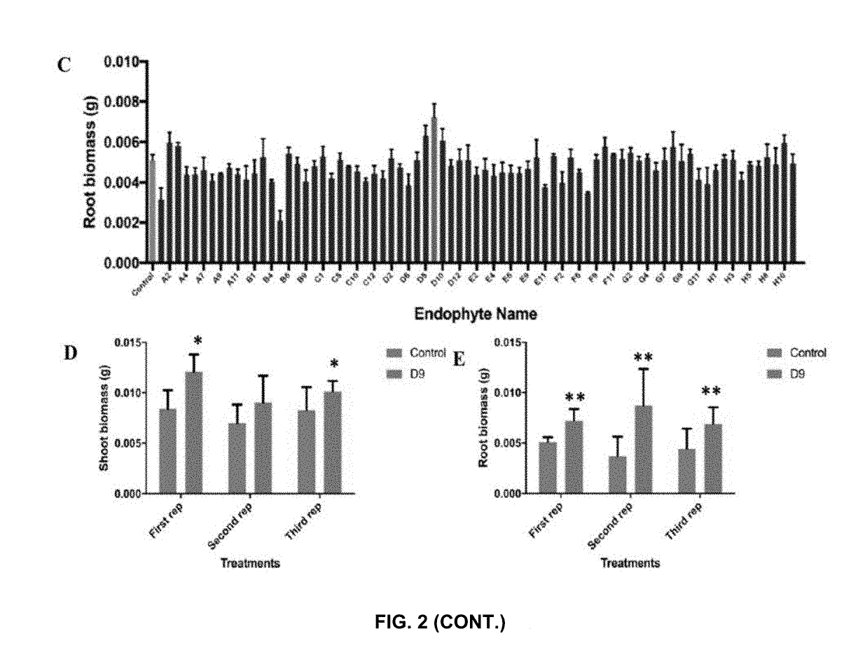

Each of the bacterial strains listed in Table 1 was screened for growth promotion of annual ryegrass. As indicated in FIGS. 2B and C, the isolated strains of bacteria exhibit varying effects on shoot biomass and root biomass of annual ryegrass. In particular, the bacterial endophyte strain 3D9 was identified to consistently have an effect on growth promotion in the absence of external nitrogen fertilizer (see FIG. 2B-E). Of the 75 bacterial endophytes that were tested 3D9 exhibited the largest increase in both shoot biomass and root biomass. As shown in Example 4, bacterial strain 3D9 also appears to promote nitrogen fixation/bioavailability in plants inoculated with the endophyte relative to controls. As shown in Example 5, inoculation with 3D9 also increases plant growth measured by an increase in mesocotyl tissue relative to controls. Bacterial strain 3D9 has been deposited as accession number 190917-01 at the International Depository Authority of Canada located at the National Microbiology Laboratory, Public Health Agency of Canada, 1015 Arlington Street, Winnipeg, Manitoba, R3E 3R2, Canada on Sep. 19, 2017.

### Definitions

The term “endophyte” as used herein refers to a class of microbial symbionts that reside within host plant seeds, roots and/or shoots, including stems, leaves, lateral buds, flowering stems and flower buds. In one embodiment, the endophyte is a bacterium, optionally bacterial strain 3D9 as described herein.

The term “inoculating a plant” as used herein refers to applying, contacting or infecting a plant (including its roots, stems, leaves or seeds) with an endophyte or a formulation comprising an endophyte. The term “inoculated plant” refers to a plant to which an endophyte or a formulation comprising an endophyte has been applied or contacted. In one embodiment, inoculating a plant refers to contacting a plant with a bacteria as described herein, wherein the bacteria colonizes the plant as an endophyte.

The term “plant” as used herein includes any member of the plant kingdom that can be colonized by a bacterial endophyte. In one embodiment, the plant is a cereal crop, optionally maize, wheat or rice, or other economically important grass species such as turfgrasses or forage annual ryegrass. In one embodiment, the plant is a fruit or vegetable. In one embodiment, the plant is selected from monocots. As used herein, the term “plant” includes parts of a plant such as roots, stems, buds, leaves, seedlings and/or seeds that can be colonized by a bacterial endophyte. In one embodiment, the plant is an agricultural plant. In one embodiment, the plant is a corn plant. In one embodiment, the plant is a seed, optionally an agricultural seed such as a corn seed.

The present disclosure contemplates the use of an “isolated” endophyte. As used herein, an isolated endophyte is an endophyte that is isolated from its native environment, and carries with it an inference that the isolation was carried out by the hand of man. An isolated endophyte is one that has been separated from at least some of the components with which it was previously associated (whether in nature or in an experimental setting).

For example, in one embodiment, the bacterial endophytes described herein may be isolated from Zea mays, Zea diploperennis or Zea nicaraguensis, as set out in Table 1. In one embodiment, the bacterial endophyte is an Enterobacter sp. endophyte isolated from Zea nicaraguensis. In one embodiment, the bacterial endophyte is strain 3D9 deposited as strain IDAC 190917-01.

The terms “improving plant growth” or “promoting plant growth” as used herein refers to an increase in size or mass of a plant or parts thereof (such as shoots, seeds and roots) compared to a control plant, or parts thereof, that has not been inoculated with the endophyte or as compared to a predetermined standard. In one embodiment, improving plant growth comprises improving nutrient status. In one embodiment, improving plant growth comprises improving nitrogen fixation and/or bioavailability.

A “synthetic combination” includes a combination of a plant, such as an agricultural plant, and an endophyte. The combination may be achieved, for example, by coating the surface of the seed of a plant, such as an agricultural plant, or plant tissues with an endophyte, or contacting a plant with a formulation comprising the endophyte, such as a spray. In one embodiment, the synthetic combination comprises a plant and a population of bacteria 3D9 which was not previously found in nature.

In one embodiment, the synthetic combination comprises endophytes that are heterologous to a seed or plant. An endophyte is considered heterologous to the seed or plant if the seed or seedling that is unmodified (e.g., a seed or seedling that is not treated with a bacterial endophyte population described herein) does not contain detectable levels of the endophyte. For example, the disclosure contemplates the synthetic combinations of plants, seeds or seedlings of plants and an endophyte population, in which the endophyte population is heterologously disposed on the exterior surface of or within a tissue of the plant, seed or seedling in an amount effective to colonize the plant. An endophyte is considered heterologously disposed on the surface or within a plant (or tissue) when the endophyte is applied or disposed on the plant in a number that is not found on that plant before application of the endophyte. For example, a bacterial endophytic population that is disposed on an exterior surface or within the seed can be an endophytic bacterium that may be associated with the mature plant, but is not found on the surface of or within the seed. As such, an endophyte is deemed heterologous or heterologously disposed when applied on the plant that either does not naturally have the endophyte on its surface or within the particular tissue to which the endophyte is disposed, or does not naturally have the endophyte on its surface or within the particular tissue in the number that is being applied.

As used herein, the term “carrier” refers to the means by which the bacterial endophyte is delivered to the target plant. Carriers that may be used in accordance with the present disclosure include oils, polymers, plastics, wood, gels, colloids, sprays, drenching means, emulsifiable concentrates and so forth. For example, in one embodiment there is provided a formulation comprising 3D9 bacteria and a carrier, optionally wherein the carrier is polyvinylpyrrolidinone (PVP).

### Bacterial Endophytes and Formulations Thereof

Endophytes, microbes that live inside a plant without causing disease, can confer beneficial traits to their host such as promoting plant growth, health, and/or vigor. As set out in Example 1, the bacterial endophytes listed in Table 1 have been isolated from seeds of diverse wild, ancient and modern genotypes of maize. Table 1 shows Genbank accession numbers for 16S rRNA sequences of the endophytes, herein incorporated by reference.

The 16S rRNA gene is widely used for the classification and identification of microbes. It is well known in the art that bacteria of the same species need not share 100% sequence identity in the 16S rRNA sequences. Accordingly, in one aspect of the disclosure, the bacterial endophyte has a 16S rRNA gene comprising a nucleotide sequence that has at least 95% sequence identity to a sequence corresponding to a genbank accession number as set forth in Table 1. In another aspect, the bacterial endophyte has a 16S rRNA gene comprising a nucleotide sequence has at least 96%, 97%^{, 97.5}%^{, 98}%, 98.5%, 99%, 99.5%, 99.9% or 100% sequence identity to the sequence corresponding to a Genbank accession number as set forth in Table 1.

In one embodiment, the bacterial endophytes comprises a 16S rDNA gene comprising a nucleotide sequence with at least 90, 95%, 96%, 97%, 98%, 99% or 100% sequence identity to 16S rDNA gene sequence of Enterobacter sp strain IDAC 190917-01, or its progeny, or mutants thereof. In one embodiment, the bacterial endophyte comprises a 16S rDNA gene comprising a nucleotide sequence of Enterobacter sp strain IDAC 190917-01. In one embodiment, the bacterial endophyte comprises a 16S rRNA sequence with at least 90, 95%, 96%, 97%, 98%, 99% or 100% sequence identity to genbank accession number JF753460.

The bacterial endophytes described herein may be grown on a substrate such as a culture medium using methods and compositions known in the art and described herein. For example, the bacterial endophytes may be cultured by shake flask cultivation, small scale or large scale cultivation (including but not limited to continuous, batch, fed-batch, or solid state cultivations) performed in suitable medium and under conditions allowing cell growth. The cultivation may take place in suitable nutrient medium comprising carbon and nitrogen sources and inorganic salts, using procedures known in the art. In one embodiment, the bacterial endophytes are cultured using Lysogeny Broth (LB) or another culture media suitable for the growth a bacterial endophyte.

In one embodiment, there is provided a formulation comprising an isolated bacterial endophyte as described herein such as bacteria 3D9. In one embodiment, the formulation comprises bacteria 3D9 and at least one of a carrier, tackifier, microbial stabilizer, fungicide, herbicide, nematicide, insecticide, plant growth regulator, rodenticide, dessicant, nutrient and culture media.

Formulations for inoculating plants with the isolated bacterial endophyte described herein are also disclosed. In one embodiment, the formulation comprises at least 10³, 10⁴, 10⁵, 10⁶, 10⁷, 10⁸ or 10⁹ CFU per ml or gram of the composition. In one embodiment, the formulation is a bacterial cell culture comprising between 10⁵ and 10⁹ CFU per ml or per gram. In one embodiment, the formulation comprises a concentration of CFU suitable for inoculating a plant using a foliar spray.

In one embodiment, the formulation comprises an isolated bacterial endophyte as described herein and a carrier. The selection of the carrier and the amount of carrier used in a composition may vary and depends on several factors including the specific use and the preferred mode of application.

The carrier can be a solid carrier or liquid carrier, and in various forms including microspheres, powders, emulsions and the like. The carrier may be any one or more of a number of carriers that confer a variety of properties, such as increased stability, wettability, or dispersability. Wetting agents such as natural or synthetic surfactants, which can be nonionic or ionic surfactants, or a combination thereof can be included in a composition of the disclosure. Water-in-oil emulsions can also be used to formulate a composition that includes the purified bacterial population.

In one embodiment, the carrier is an agricultural carrier such as soil or a plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers.

In one embodiment, the composition comprises a suspension of the isolated bacterial endophyte and a seed coating agent as carrier. Optionally, the seed coating agent is polyvinyl pyrrolidine (PVP).

In one embodiment, the formulation can include a tackifier or adherent. Such agents are useful for combining the bacterial population of the disclosure with carriers that can contain other compounds (e.g., control agents that are not biologic), to yield a coating composition. Such compositions help create coatings around the plant or seed to maintain contact between the microbe and other agents with the plant or plant part. In one embodiment, adherents are selected from the group consisting of: alginate, gums, starches, lecithins, formononetin, polyvinyl alcohol, alkali formononetinate, hesperetin, polyvinyl acetate, cephalins, Gum Arabic, Xanthan Gum, Mineral Oil, Polyethylene Glycol (PEG), Polyvinyl pyrrolidone (PVP), Arabino-galactan, Methyl Cellulose, PEG 400, Chitosan, Polyacrylamide, Polyacrylate, Polyacrylonitrile, Glycerol, Triethylene glycol, Vinyl Acetate, Gellan Gum, Polystyrene, Polyvinyl, Carboxymethyl cellulose, Gum Ghatti, and polyoxyethylene-polyoxybutylene block copolymers. The formulation can also contain a surfactant. Non-limiting examples of surfactants include nitrogen-surfactant blends.

In certain cases, the formulation includes a microbial stabilizer. Such an agent can include a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the bacterial population used, and should promote the ability of the microbial population to survive application on the seeds and to survive desiccation. Examples of suitable desiccants include one or more of trehalose, sucrose, glycerol, and methylene glycol. Other suitable desiccants include, but are not limited to, non-reducing sugars and sugar alcohols (e.g., mannitol or sorbitol).

In some cases, it is advantageous for the formulation to contain agents such as a fungicide, an antibacterial agent, an herbicide, a nematicide, an insecticide, a plant growth regulator, a rodenticide, and/or a nutrient. Such agents are ideally compatible with the agricultural seed or seedling onto which the formulation is applied (e.g., it should not be deleterious to the growth or health of the plant). Furthermore, the agent is ideally one which does not cause safety concerns for human, animal or industrial use (e.g., no safety issues, or the compound is sufficiently labile that the commodity plant product derived from the plant contains negligible amounts of the compound).

In one embodiment of the disclosure, the composition is in a fluid form suitable for spray application such as for foliar application or seed coating. In the liquid form, for example, solutions or suspensions, the bacterial endophytic populations of the present disclosure can be mixed or suspended in water or in aqueous solutions. Suitable liquid diluents or carriers include water, aqueous solutions, petroleum distillates, or other liquid carriers.

In another embodiment, said composition is in a paste-like form. In still another embodiment, the composition is in a substantially dry and powdered form for dusting. Solid compositions can be prepared by dispersing the bacterial endophytic populations of the disclosure in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

The composition is optionally applied as a spray. In another embodiment, the composition is applied to seeds, as a seed coating. In yet another embodiment, the composition is applied both as a spray and a seed coating. In one embodiment, the composition or formulation described herein is a foliar spray. In one embodiment, the methods described herein include inoculating a plant using a foliar spray comprising a bacterial endophyte.

Also provided are synthetic combinations of the isolated bacterial endophyte described herein in association with a plant. In one embodiment, the bacterial endophytes reside within the seeds, roots, shoots, stems and/or leaves of the plant as an endophyte. Optionally, the plant may be a plant seed or seedling. In one embodiment, the bacterial endophyte is heterologous to the microbial population of the plant. For example, synthetic combination may be a bacterial strain such as 3D9 as described herein which has been artificially inoculated on a plant that does not naturally harbor or contain the bacterial endophyte.

In one embodiment, the endophyte is present in the synthetic combination in an amount effective to provide a benefit to the seeds or seedlings or plants derived from the seeds or seedlings. In one embodiment, the bacterial endophyte is present in the synthetic combination at a concentration of at least 10³, 10⁴, 10⁵, 10⁶, 10⁷, 10⁸ or 10⁹ colony-forming units (CFU) per seed, optionally between about 10⁴ and 10⁹ CFU per seed or between about 10⁴ and 10⁷ CFU per seed.

In another aspect, there is provided a method for preparing an agricultural seed composition comprising contacting the surface of a plurality of seeds with a formulation comprising a bacterial endophyte as described herein. In one embodiment, the endophyte is present in the synthetic combination in an amount effective to provide a benefit to the seeds or plants derived from the seeds. For example, in one embodiment the benefit is increased plant growth. In one embodiment, the benefit is nitrogen fixation and/or bioavailability. In one embodiment, the endophyte is heterologous to the seeds. In one embodiment, the bacterial endophyte is present in the synthetic combination at a concentration sufficient to increase or promote plant growth in a plant produced from the seed relative to a plant produced from a seed that is not in association with the bacterial endophyte grown under the same conditions.

In another embodiment, there is provided an article of manufacture or kit that includes packaging material, plant seeds within the packaging material, and at least one species of bacterial endophyte associated with the seeds. In one embodiment, the article of manufacture comprises a synthetic combination as described herein. In one embodiment, at least 100, 200, 500, 750, 1000, 1250, 1500, 1750, 2000, 3000, 4000 or 5000 seed weight amount of seeds are disposed within the packaging material. Optionally, a carrier may also be disposed within the article of manufacture in association with the seeds.

### Methods and Uses of the Bacterial Endophytes

In one aspect, it has been shown that the bacterial endophytes described herein exhibit one or more characteristics that are beneficial for plants associated with the bacterial endophytes. For example, as shown in Example 2, the bacterial endophytes identified in Table 1 may confer beneficial properties such as improving the growth or yield of a plant and improving nutrient acquisition. The bacterial strain 3D9 has been demonstrated to be surprisingly effective at improving the growth of plants.

Accordingly, in one embodiment there is provided a method of treating a plant to promote growth comprising contacting the plant with an isolated culture of 3D9 bacteria or a formulation comprising 3D9 bacteria as described herein. Various methods known in the art may be used to contact the plant with the bacterial endophyte such that the plant is inoculated with the bacterial endophyte to confer a benefit to the plant. For example, in one embodiment the method comprises contacting the plant with the bacterial endophyte by applying a spray, a mist, a dip, or a nutrient solution to the plant that includes the bacterial endophyte. In one embodiment, the method comprises contacting a plant with a bacterial endophyte that is heterologous to the plant. The bacterial endophytes may be applied to the roots or shoots of the plant, or to young germinated seedlings, or to ungerminated or germinated seeds. In one embodiment, the bacterial endophytes may be sprayed onto the plant, either in a liquid formulation or as a powdered inoculum. Alternatively, a composition comprising the bacterial endophytes can be applied by drip or other irrigation system.

In one embodiment, there is provided a method of treating a plant to promote nitrogen fixation/bioavailability comprising contacting the plant with an isolated culture of 3D9 bacteria or a formulation comprising 3D9 bacteria as described herein.

Also provided is a method for the production of a bacterial endophyte culture. In one embodiment, the method comprises inoculating a substrate with bacteria as described herein such as 3D9 and incubating the substrate under conditions suitable for bacterial growth. It is therefore possible to expand a bacterial culture described herein by many orders of magnitude in order to produce a culture of a bacterial endophyte suitable for use in a formulation and/or method as described herein. For example, the bacterial endophytes may be cultured by shake flask cultivation, small scale or large scale cultivation (including but not limited to continuous, batch, fed-batch, or solid state cultivations) performed in suitable medium and under conditions allowing bacterial cell growth. The cultivation may take place in suitable nutrient medium comprising carbon and nitrogen sources and inorganic salts, using procedures known in the art. In one embodiment, the bacterial endophytes are cultured using Lysogeny Broth (LB) or another culture media suitable for bacterial growth. In one embodiment, the methods include culturing in LB liquid media with shaking, optionally at about 250 rpm, at about 37° C.

The above disclosure generally describes the present application. A more complete understanding can be obtained by reference to the following specific examples. These examples are described solely for the purpose of illustration and are not intended to limit the scope of the application.

Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present application:

### EXAMPLES

### Example 1: Selecting a Turfgrass Genotype for Use as a Model System

### Summary

Endophytes are microbes that colonize plant internal tissues without causing disease. Herein described is a model system for selecting a turfgrass genotype for screening maize endophytes for growth promotion.

### Materials and Methods

### Seed and Endophyte Materials

Annual ryegrass (Lolium multiflorum) variety Annuity was obtained from Seed Research of Oregon (Oregon, USA), while perennial ryegrass (Lolium perenne), Kentucky bluegrass (Poa pratensis), Bermuda grass (Cynodon dactylon) (Southern Stay), and tall fescue (Lolium arundinaceum) were courtesy of the Guelph Turfgrass Institute (Guelph, Canada). The bacterial endophyte collection (Table 1) was previously isolated from seeds of diverse wild, ancient and modern genotypes of maize (Johnston-Monje and Raizada 2011a).

TABLE 1
List of maize endophytes used in this study with their taxonomic
predictions, Genbank accession numbers for 16S rRNA sequences, and
original host species.

| | | Genbank | |
|---|---|---|---|
| ID | Endophyte genus | accession | Host species |
| 3A1 | Pantoea | JF753401 | Zea mays ssp mays |
| | | | (inbred B73) |
| 3A2 | Enterobacter | JF753409 | Zea mays ssp mays (landrace |
| | | | chapalote) |
| 3A3 | Rhodococcus | JF753402 | Zea mays ssp mays |
| | | | (inbred B73) |
| 3A4 | Pseudomonas | JF753403 | Zea mays ssp mays |
| | | | (inbred B73) |
| 3A5 | Pantoea | JF753404 | Zea mays ssp mays (landrace |
| | | | Bolita) |
| 3A7 | Enterobacter | JF753407 | Zea mays ssp mays (landrace |
| | | | Bolita) |
| 3A8 | Enterobacter | JF753410 | Zea mays ssp mays (landrace |
| | | | Bolita) |
| 3A9 | Enterobacter | JF753411 | Zea mays ssp mays (landrace |
| | | | chapalote) |
| 3A10 | Hafnia | JF753412 | Zea mays ssp mays (landrace |
| | | | chapalote) |
| 3A11 | Epascherichia coli | JF753413 | Zea mays ssp mays (landrace |
| | | | chapalote) |
| 3A12 | Burkholderia | JF753414 | Zea mays ssp mays (landrace |
| | | | chapalote) |
| 3B1 | Enterobacter | JF753415 | Zea mays ssp mays (landrace |
| | | | chapalote) |
| 3B3 | Micrococcus | JF753417 | Zea mays ssp mays (landrace |
| | | | chapalote) |
| 3B4 | Pantoea | JF753491 | Zea mays ssp mays (landrace |
| | | | chapalote) |
| 3B5 | Enterobacter | JF753408 | Zea mays ssp mays (landrace |
| | | | chapalote) |
| 3B6 | Pseudomonas | JF753420 | Zea mays ssp mays (landrace |
| | | | chapalote) |
| 3B7 | Enterobacter | JF753421 | Zea mays ssp mays (landrace |
| | | | chapalote) |
| 3B9 | Escherichia coli | JF753424 | Zea mays ssp mays (landrace |
| | | | Cristalino) |
| 3B10 | Staphylococcus | JF753425 | Zea mays ssp mays (landrace |
| | | | Cristalino) |
| 3C1 | Enterobacter | JF753429 | Zea diploperennis |
| 3C7 | Enterobacter | JF753435 | Zea diploperennis |
| 3C8 | Pseudomonas | JF753436 | Zea diploperennis |
| 3C9 | Pseudomonas | JF753440 | Zea diploperennis |
| 3C10 | Enterobacter | JF753441 | Zea diploperennis |
| 3C11 | Burkholderia | KP455296 | Zea diploperennis |
| 3C12 | Enterobacter | JF753449 | Zea diploperennis |
| 3D1 | Enterobacter | JF753450 | Zea diploperennis |
| 3D2 | Pantoea | JF753451 | Zea mays ssp mays (landrace |
| | | | Gaspe Flint) |
| 3D5 | Arthrobacter | JF753454 | Zea mays ssp mays (landrace |
| | | | Gaspe Flint) |
| 3D6 | Cellulomonas | JF753455 | Zea mays ssp mays (landrace |
| | | | Gaspe Flint) |
| 3D7 | Pantoea | JF753456 | Zea mays ssp mays (landrace |
| | | | Jala) |
| 3D8 | Arthrobacter | JF753458 | Zea nicaraguensis |
| 3D9 | Enterobacter | JF753460 | Zea nicaraguensis |
| 3D10 | Enterobacter | JF753461 | Zea nicaraguensis |
| 3D11 | Pantoea | JF753462 | Zea nicaraguensis |
| 3D12 | Enterobacter | JF753469 | Zea nicaraguensis |
| 3E1 | Enterobacter | JF753463 | Zea nicaraguensis |
| 3E2 | Stenotrophomonas | JF753464 | Zea nicaraguensis |
| 3E3 | Stenotrophomonas | JF753465 | Zea nicaraguensis |
| 3E4 | Klebsiella | JF753466 | Zea nicaraguensis |
| 3E5 | Citrobacter | JF753467 | Zea nicaraguensis |
| 3E6 | Pantoea | JF753468 | Zea nicaraguensis |
| 3E7 | Paenibacillus | JF753470 | Zea nicaraguensis |
| 3E9 | Microbacterium | JF753473 | Zea nicaraguensis |
| 3E10 | Paenibacillus | JF753475 | Zea nicaraguensis |
| 3E11 | Pantoea | JF753477 | Zea nicaraguensis |
| 3E12 | Pantoea | a JF753478 | Zea mays ssp mexicana |
| 3F2 | Pseudomonas | JF753483 | Zea mays ssp mexicana |
| 3F3 | Pantoea | JF753484 | Zea mays ssp mexicana |
| 3F6 | Pseudomonas | JF753488 | Zea mays ssp mexicana |
| 3F7 | Pantoea | JF753489 | Zea mays ssp mexicana |
| 3F9 | Burkholderia | JF753492 | Zea mays ssp mays (landrace |
| | | | mixteco) |
| 3F10 | Burkholderia | JF753493 | Zea mays ssp mays (landrace |
| | | | mixteco) |
| 3F11 | Enterobacter | JF753476 | Zea nicaraguensis |
| 3G1 | Methylobacterium | JF753495 | Zea mays ssp mays (landrace |
| | | | nal-Tel) |
| 3G2 | Paenibacillus | JF753496 | Zea mays ssp mays (landrace |
| | | | nal-Tel) |
| 3G3 | Stenotrophomonas | JF753490 | Zea mays ssp mays (landrace |
| | | | nal-Tel) |
| 3G4 | Pantoea | JF753497 | Zea mays ssp parviglumis |
| 3G6 | Stenotrophomonas | JF753499 | Zea mays ssp parviglumis |
| 3G7 | Pantoea | JF753500 | Zea mays ssp parviglumis |
| 3G8 | Klebsiella | JF753501 | Zea mays ssp parviglumis |
| 3G9 | Paenibacillus | JF753471 | Zea nicaraguensis |
| 3G10 | Klebsiella | KU214888 | Zea mays ssp parviglumis |
| 3G11 | Paenibacillus | JF753509 | Zea mays ssp parviglumis |
| 3H1 | Bacillus | JF753511 | Zea mays ssp mays (inbred |
| | | | Pioneer 3751) |
| 3H2 | Deinococcus | JF753512 | Zea mays ssp mays (inbred |
| | | | Pioneer 3751) |
| 3H3 | Deinococcus | JF753513 | Zea mays ssp mays (inbred |
| | | | Pioneer 3751) |
| 3H4 | Rhodococcus | JF753514 | Zea mays ssp mays (inbred |
| | | | Pioneer 3751) |
| 3H5 | Microbacterium | JF753515 | Zea mays ssp mays (inbred |
| | | | Pioneer 3751) |
| 3H6 | Bacillus | JF753516 | Zea mays ssp mays (inbred |
| | | | Pioneer 3751) |
| 3H8 | Bacillus | JF753518 | Zea mays ssp mays (inbred |
| | | | Pioneer 3751) |
| 3H9 | Methylobacterium | JF753519 | Zea mays ssp mays (inbred |
| | | | Pioneer 3751) |
| 3H10 | Bradyrhizobium | JF753520 | Zea mays ssp mays (landrace |
| | | | Tuxpeno) |
| 3H11 | Rhodococcus | KU214889 | Zea mays ssp mays (inbred |
| | | | Pioneer 3751) |

### Selecting a Turfgrass Genotype for Use as a Model System for Nutrient Promoting Endophytes

To select a model turfgrass, five turfgrass genotypes were evaluated for four criteria:

Germination, Uniformity and Rate of Growth.

Fifteen seeds per genotype were germinated on wet paper towels (water only) in Petri dishes, and growth was observed for 10 days. There were three replicate dishes per genotype. Seeds were also germinated on 0.5 strength Murashige & Skoog (MS) medium in glass tubes and observed for growth over three weeks. Each glass tube (15 cm×25 cm, C5916, Sigma, USA), capped (C5791, Sigma, USA) contained 15 ml of autoclaved 0.5 strength MS (pH 5.8), consisting of (per L): half-strength modified basal MS salt (M571, Phytotech, USA), 250 μl nicotinic acid (1 mg/ml), 500 μl pyridoxine HCl (0.5 mg/ml), 5 ml thiamine HCl (100 mg/l), 500 μl glycine (2 mg/ml) and 2 g Phytagel (P8169, Sigma, USA) in double distilled water. To solidify Phytagel, 0.166 g/l CaCl₂) and 90 mg/l MgSO₄ were added.

Surface Sterilization Efficiency.

Two protocols were used: (Method 1) washing seeds in 70% ethanol for 1 min, then washing in bleach for 10 min and six washes with water; (Method 2) washing seeds in 70% ethanol for 1 min, then washing in bleach for 20 min and six washes with water. Twenty microliters from the last wash were spotted on R-2A agar (17209, Sigma, USA) to test for microbial growth.

Growth on Different Media:

Annual ryegrass and tall fescue seeds were surface sterilized using Sterilization Method 2 then grown on four different media: 1.5% bacto-agar (DF0140, Fisher), 1.5% R-2A agar (17209, Sigma, USA), sand (15 g sand and 4 ml of water), and Phytagel (0.5 79 strength MS). There were three seeds/tube and three replicate tubes/treatment. Growth was observed over three weeks.

Responsiveness to Mineral Nutrients:

Annual ryegrass and tall fescue seeds were surface sterilized using Sterilization Method 2 and grown at three different nutrient concentrations (0 84 MS, 0.1 strength MS and 0.5 strength MS). There were four tubes/treatment and three seeds/tube. After one month, plants were removed, rinsed, and air-dried for 20 min. Shoots and roots were dissected and weighed. The 0 strength MS medium (pH 5.8) consisted of (per L): 2 g Phytagel, 0.33 g/l CaCl₂) and 180 mg/l MgSO₄ in double distilled water.

### Results and Discussion

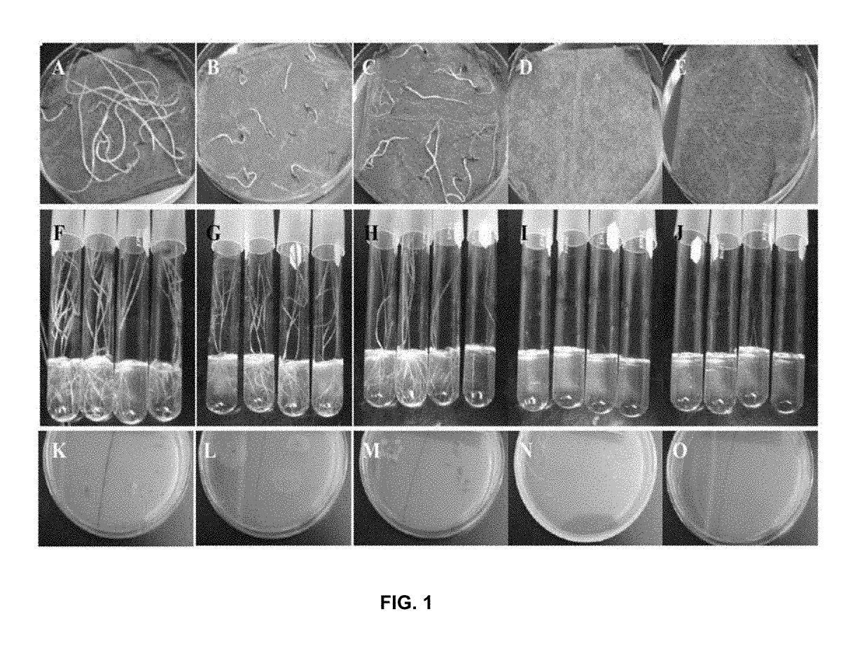

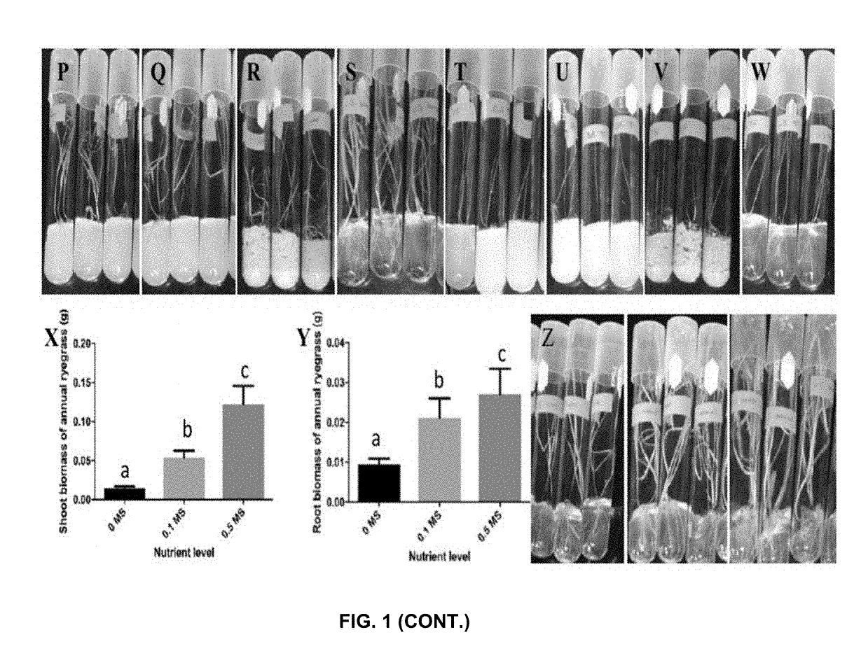

Germination, uniformity and rate of growth: Five turfgrass genotypes (annual ryegrass, perennial ryegrass, tall fescue, Bermuda grass, Kentucky bluegrass) were tested for germination and growth rate on paper towels and in tubes with 0.5 strength MS media. Annual ryegrass, perennial ryegrass and tall fescue were fast growing while Bermuda grass and Kentucky bluegrass were slow growing (FIG. 1A-J, Table 2).

Efficiency of Surface Sterilization:

As endophyte assays often involve coating of surface sterilized seeds, the ability to efficiently surface sterilize the turfgrass genotypes was evaluated using two protocols. Washing seeds in 70% ethanol for 1 min, then washing in bleach for 20 min and six washes with water (Method 2), was efficient at surface sterilizing all genotypes except perennial ryegrass (FIG. 1K-O, Table 2).

Growth on Different Media:

The ability of plants to grow on different media offers distinct opportunities. The two promising turf genotypes that passed the above criteria (annual ryegrass, tall fescue) were grown on four different media (bacto-agar, R-2A agar, sand, Phytagel). Both genotypes grew well on all media types but Phytagel had the advantage of being transparent allowing visualizing plant root growth without removing plants from tubes (FIG. 1P-W).

Responsiveness to Mineral Nutrition:

To use a turfgrass as a model, it must be responsive to increasing nutrients. Annual ryegrass and tall fescue were grown at three different nutrient 125 concentrations (0 MS, 0.1 strength MS, 0.5 strength MS). After 1 month, annual ryegrass showed significant differences in both shoot and root biomass on all three nutrient concentrations (FIG. 1X-Z) while tall fescue only showed a significant difference in shoot biomass which was limited to 0 MS versus 0.5 strength MS.

TABLE 2
Summary of tests conducted to select a turfgrass genotype as a model system.

| Selection | | | | | | | | |
|---|---|---|---|---|---|---|---|---|
| criteria | Turfgrass species | Replicate 1 | | Replicate 2 | | Replicate 3 | | Conclusion |
| Percentage seed | Annual ryegrass | 87% | | 80% | | 93% | | Acceptable |
| germination | Perennial ryegrass | 67% | | 40% | | 47% | | Acceptable |
| after 10 days | Tall fescue | 80% | | 60% | | 33% | | Acceptable |
| | Kentucky bluegrass | 0 | | 0 | | 0 | | Excluded |
| | Bermuda grass | 13% | | 0 | | 0 | | Excluded |
| Rate of growth | Annual ryegrass | +++ | | +++ | | +++ | | Acceptable |
| after 3 weeks | Perennial ryegrass | + | | + | | + | | Acceptable |
| (+++ = fastest) | Tall fescue | + | | + | | + | | Acceptable |
| | Kentucky bluegrass | − | | − | | − | | Excluded |
| | Bermuda grass | − | | − | | − | | Excluded |
| | | Method 1 | Method 2 | Method 1 | Method 2 | Method 1 | Method 2 | |
| Microbial | Annual ryegrass | − | − | − | − | − | − | Selected |
| contamination | Perennial ryegrass | + | + | + | − | + | + | Excluded |
| observed after | Tall fescue | + | + | + | − | + | − | Acceptable |
| seed sterilization | Kentucky bluegrass | − | − | − | − | − | − | Acceptable |
| | Bermuda grass | + | − | − | + | − | − | Acceptable |

### Example 2: Use of Annual Ryegrass as an in Planta Model System to Screen Maize Endophytes

Annual ryegrass was used as an in planta model system to screen maize endophytes. A number of endophytes, including Enterobacter sp strain 3D9 (i.e. strain IDAC 190917-01), were found to promote the growth of shoot or root biomass.

### Materials and Methods

### Screening a Collection of Maize Endophytes for Growth Promotion of Annual Ryegrass in the Absence of Nitrogen

A modified 0.5 strength MS medium was prepared as described in Example 1 except that the modified MS basal salt mixture contained no nitrogen (M531, Phytotech, USA). Maize endophytes were coated onto annual ryegrass seeds as previously described (Shehata et al. 2016b, hereby incorporated by reference in its entirety). Seven seeds were germinated per tube and each endophyte was tested in triplicate. After four weeks, plants were removed, rinsed and air-dried for 20 min. Shoots and roots were dissected and weighed as pools per tube, then divided by the number of plants to calculate the mean weight per tube. Positive candidates were rescreened in two additional trials with seven plants/tube and seven replicate tubes per endophyte.

Taxonomic Identification of Maize Endophytes with Growth Promotion Ability

Taxonomic analysis employed 16S rRNA universal primers 799f and 1492r as previously described (Shehata et al. 2016a, hereby incorporated by reference in its entirety).

### Statistical Analysis and Graphs

Microsoft Excel 2011 and GraphPad Prism 7 were used for graphing and for statistical analysis (Student t-test).

### Results and Discussion

Based on the above criteria, the annual ryegrass tube system was selected as a high-throughput model to test nutrient activities of cereal endophytes. Annual ryegrass is an economically important crop used as cattle feed and for remediation of manure phosphate. There is significant interest in discovering endophytes that allow crops including maize to grow with reduced nitrogen fertilizer (Johnston-Monje and Raizada 2011b). Thus, this new model system was used to screen a maize bacterial endophyte collection (Johnston-Monje and Raizada 2011a) for plant growth promotion in the absence of nitrogen. Out of 75 endophytes tested (FIG. 2A-C), strain 3D9 was found to consistently promote root biomass in three independent trials (p=0.04, 0.007, 0.03). The growth promotion effect was weaker but promising for shoots (p=0.07, 0.12, 0.08) (FIGS. 2D and E). Based on 16S rRNA sequencing, strain 3D9 was found to most closely resemble Enterobacter (99% similarity). The growth promotion activity may be attributed to biological nitrogen fixation (de Souza et al. 2015; Santi et al. 2013), root scavenging of secreted nitrogen metabolites normally used to support the rhizosphere (Tkacz and Poole 2015), and/or improved recycling of nitrogen metabolites from senescing tissues to growing tissues. The results demonstrate that the annual ryegrass tube 146 system has the potential to rapidly identify potentially important cereal endophytes.

### Example 3: Methods Involving Annual Ryegrass to Screen Endophytes that Promote Other Nutrients

Annual ryegrass could be used to screen endophytes that promote other nutrients including phosphorous as it is a phosphorous-hyperaccumulator, or for traits such as root and/or shoot phenotyping caused by microbial production of phytohormones. The transparency of the Phytagel based media enables non-destructive monitoring of root growth. In addition, annual ryegrass and other turfgrasses hold potential as model systems to screen microbes with anti-pathogen activities when the target pathogen affects both the cereal as well as the turfgrass. Creeping bentgrass may be useful for screening for maize endophytes that suppress the fungal pathogen Rhizoctonia solani, which affects both turfgrass and maize.

### Example 4: Effects of Inoculating Plants with Endophyte Strain 3D9 on Nitrogen Status

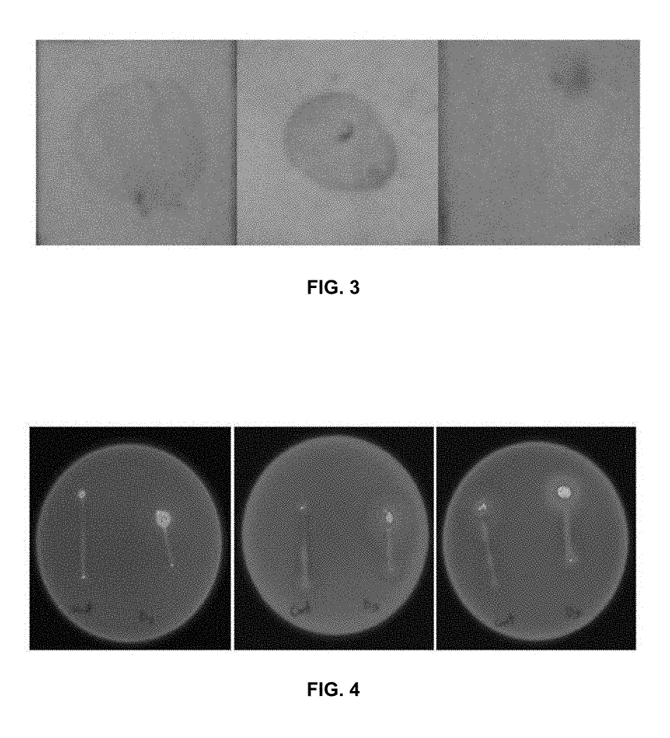

A potential mechanism of growth promotion under no nitrogen condition is nitrogen fixation. Nitrogen fixation is the process of converting atmospheric nitrogen (N₂), which is unusable by plants, to ammonia (NH₃), which can be used by plants. This process is mediated by the nitrogenase enzyme. Dot blot hybridization showed that strain 3D9 expressed the nifH gene, one of the structural genes for nitrogenase (FIG. 3).

To evaluate the effects of strain 3D9 on nitrogen status of corn plants, 3D9 was applied as seed coats. After three weeks of growth in growth pouches, plants were harvested, cleaned, dissected into root, stem, and shoot. The tissues were freeze thawed to promote leakage of internal nitrogen metabolites including the amino acid glutamine. Then, the different parts were incubated on GlnLux agar plates along with parts from control uninoculated plants. A CCD camera was used to take images over a time course. GlnLux is a biosensor that emits photons of light in response to the presence of exogenous glutamine that is critical for nitrogen assimilation in maize. After six hours of incubation, there was an obvious difference in GlnLux signal, with 3D9 inoculated plants showing a stronger signal compared to uninoculated plants (FIG. 4). The stronger signal indicates that 3D9 inoculated plants accumulated more glutamine. The higher glutamine content indicates a difference in nitrogen status.

Since the plants were grown in the absence of external nitrogen, the higher glutamine content can mean that 3D9 is i) converting atmospheric nitrogen gas into ammonia (i.e. nitrogen fixation), ii) remobilizing existing organic nitrogen reserved in the plant, iii) promoting better root scavenging of nitrogen released by roots, and/or iv) influencing other alterations in nitrogen metabolism. The presence of nifH gene in strain 3D9 shows that the impact on nitrogen status by this strain may at least in part mediated by nitrogen fixation.

### Example 5: Inoculation with 3D9 Improves Nitrogen Fixation/Bioavailability and Plant Growth

Additional experiments were performed using GlnLux agar plates to test for the effect of 3D9 on nitrogen fixation and/or internal nitrogen bioavailability. The methodology of the GlnLux assay is described in detail in Tessaro et al. (2012), hereby incorporated by reference. More recently its reliability as an indicator of nitrogen fixation (in legumes) was demonstrated by Thilakarathna and Raizada (2018), hereby incorporated by reference.

Strain 3D9 was inoculated onto corn seeds and then germinating seedlings. Following freeze-thawing, the samples were placed on agar containing GlnLux biosensor cells. The freeze-thaw causes Gln from corn tissues to leek onto the agar which switches on the biosensor which emits luminescence which is then detected using a CCD photon capture camera.

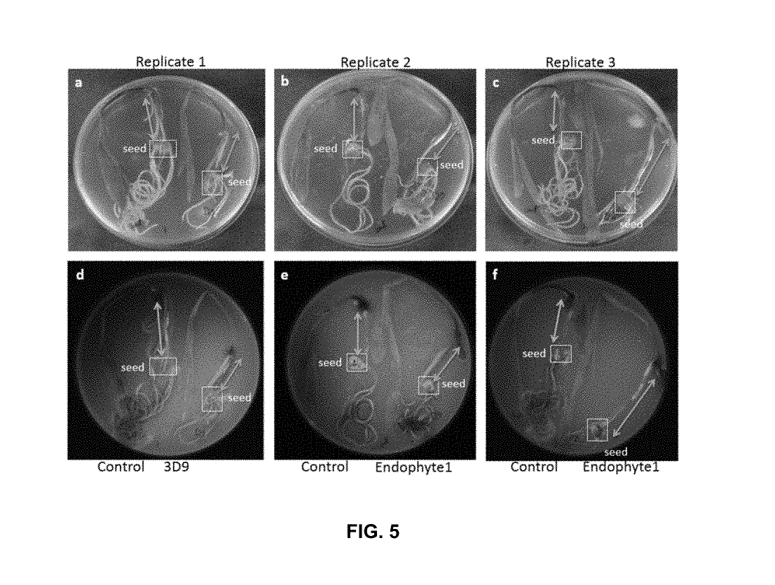

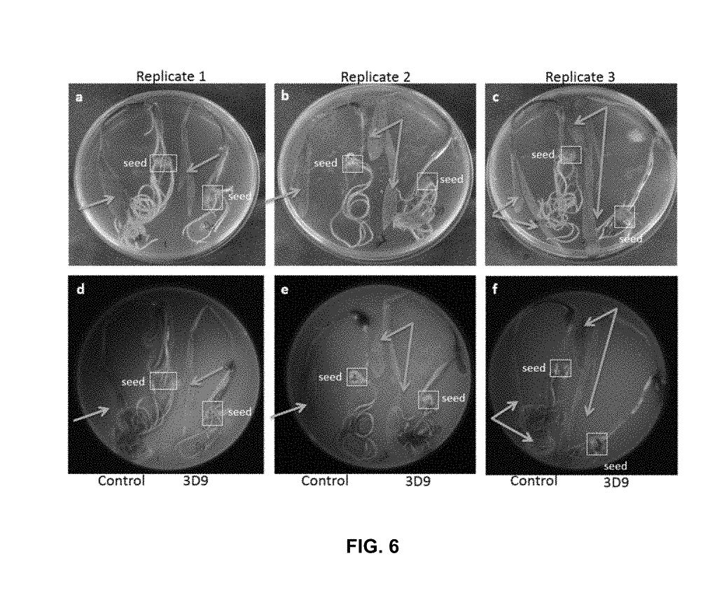

As shown in FIGS. 5 and 6, corn stems (mesocotyl, right side arrows of each replicate in FIG. 5) and leaves (arrows in FIG. 6) inoculated with 3D9 were observed to emit more light relative to controls (left side arrows in FIGS. 5 and 6) indicating higher levels of the amino acid glutamine (Gin). When nitrogen is added to corn or recycled internally, it is first incorporated into Gln which is an essential transport form of nitrogen in corn.

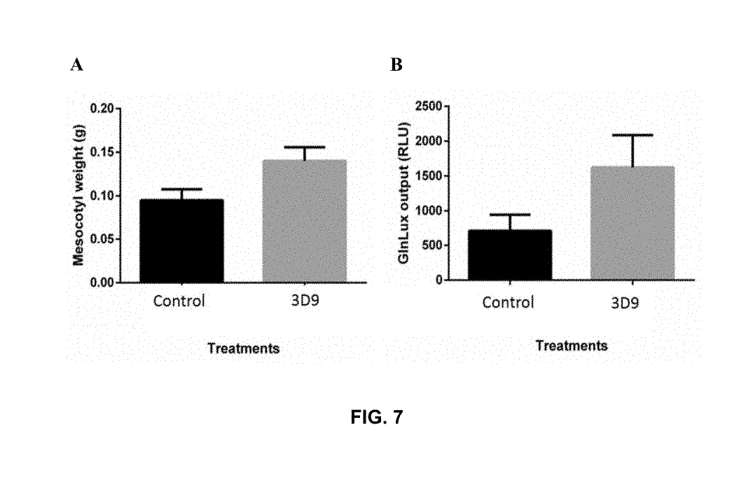

FIG. 7 quantifies the results from FIG. 5 and also shows that corn mesocotyl tissues inoculated with 3D9 had a greater mass relative to controls, demonstrating a positive effect on plant growth, along with greater GlnLux output. Accordingly, 3D9 improves plant growth and bioavailable nitrogen in plants such as corn.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents, patent applications, genbank accession numbers, and biological deposits are herein incorporated by reference in their entirety to the same extent as if each individual publication, patent, patent application, genbank accession number, or biological deposit was specifically and individually indicated to be incorporated by reference in its entirety.

### REFERENCES CITED

de Souza R, Ambrosini A, Passaglia L M P (2015) Plant growth-promoting bacteria as inoculants in agricultural soils. Genet Mol Biol 38:401-419 doi:10.1590/s1415-475738420150053
Johnston-Monje D, Raizada M N (2011a) Conservation and diversity of seed associated endophytes in Zea across boundaries of evolution, ethnography and ecology. PLoS ONE 6:e20396 doi:10.1371/journal.pone.0020396
Johnston-Monje D, Raizada M N (2011b) Integration of biotechnologies—plant and endophyte relationships: nutrient management. In: Moo-Young M (ed) Comprehensive Biotechnology, second edition, vol 4. Elsevier, pp 713-727
Pliego C, Ramos C, Vicente A, Cazorla F (2011) Screening for candidate bacterial biocontrol agents against soilborne fungal plant pathogens. Plant Soil 340:505-520 doi:10.1007/s11104-010-0615-8
Rey T, Schornack S (2013) Interactions of beneficial and detrimental root-colonizing 210 filamentous microbes with plant hosts. Genome Biology 14:121-121 doi:10.1186/gb-2013-14-6-121
Santi C, Bogusz D, Franche C (2013) Biological nitrogen fixation in non-legume plants. Ann Bot 111:743-767 doi:10.1093/aob/mct048
Shehata H R, Lyons E M, Jordan K S, Raizada M N (2016a) Bacterial endophytes from wild and 226 ancient maize are able to suppress the fungal pathogen Sclerotinia homoeocarpa. J Appl Microbiol 120:756-769 doi:10.1111/jam.13050
Shehata H R, Lyons E M, Jordan K S, Raizada M N (2016b) Relevance of in vitro agar based screens to characterize the anti-fungal activities of bacterial endophyte communities. BMC Microbiol 16:1-7 doi:10.1186/s12866-016-0623-9
Tessaro M J, Soliman S S M, Raizada M N. (2012) Bacterial whole-cell biosensor for glutamine with applications for quantifying and visualizing glutamine in plants. Appl. Environ. Microbiol. 2012, 78(2):604.
Thilakarathna M S and Raizada M N (2018) Visualizing glutamine accumulation in root systems involved in the legume-rhizobia symbiosis by placement on agar embedded with companion biosensor cells. Phytobiomes Journal. 2018, Volume 2, Number 3 pp. 117-128
Tkacz A, Poole P (2015) Role of root microbiota in plant productivity. J Exp Bot 66:2167-2175 doi:10.1093/jxb/erv157
Wani Z, Ashraf N, Mohiuddin T, Riyaz-Ul-Hassan S (2015) Plant-endophyte symbiosis, an ecological perspective. Appl Microbiol Biot 99:2955-2965 doi:10.1007/s00253-015-6487-3

1. An isolated culture of bacteria deposited as strain IDAC 190917-01.

2. A formulation comprising the isolated culture of claim 1, and at least one of a carrier, tackifier, microbial stabilizer, fungicide, herbicide, nematicide, insecticide, plant growth regulator, rodenticide, dessicant, nutrient and culture media.

3. The formulation of claim 2, wherein the carrier comprises a surfactant or seed coating agent.

4. The formulation of claim 2, wherein the formulation is in liquid form or in solid form.

5. The formulation of claim 2, wherein the formulation is in liquid form and has a concentration of the bacteria at least 10⁵, 10⁶, 10⁷ or 10⁸ CFU/m L.

6. The formulation of claim 4, wherein the formulation is in solid form.

7. The formulation of claim 2, wherein the carrier is polyvinylpyrrolidinone (PVP).

8. A synthetic combination comprising bacteria deposited as strain IDAC 190917-01 in association with a plant, wherein the bacteria is heterologous to the plant.

9. The synthetic combination of claim 8, wherein the plant is an agricultural plant such as a fruit or vegetable.

10. The synthetic combination of claim 9, wherein the agricultural plant is corn.

11. The synthetic combination of claim 8, wherein the bacteria is in association with a seed or seedling.

12. The synthetic combination of claim 11, wherein the bacteria is in association with the seed and the concentration of the bacteria is at least 10³ colony-forming units (CFU) per seed.

13. The synthetic combination of claim 11, wherein the concentration of the bacteria is between about 10⁴ and 10⁷ CFU per seed.

14. The synthetic combination of claim 11, wherein the seed is corn seed.

15. A method of treating a plant to promote growth comprising contacting the plant with a bacteria deposited as strain IDAC 190917-01, wherein the bacteria colonizes the plant as an endophyte.

16. The method of claim 15, wherein contacting the plant with the isolated culture or the formulation comprises applying a spray, a mist, a dip, or a nutrient solution to the plant.

17. The method of claim 15, wherein the plant is a plant seed or seedling.

18. The method of claim 15, wherein contacting the plant with the bacteria increases biomass of the plant relative to a control plant.

19. The method of claim 15, wherein contacting the plant with the bacteria improves bioavailable nitrogen in the plant relative to a control plant.

20. A method for the production of a bacterial endophyte culture, the method comprising:
inoculating a substrate with the isolated culture of claim 1; and
incubating the substrate under conditions suitable for bacterial growth.
